(19) United States
(12) Patent Application Publication
(10) Pub. No.: US 2009/0137643 A1
(43) Pub. Date: May 28, 2009

(54) Methods for Inhibiting or Reversing Tau Filament Fibrillization

(76) Inventors: Jeff Kuret, Dublin, OH (US); Sam Khatami, Highland Park, IL (US)

Correspondence Address: FITCH EVEN TABIN AND FLANNERY, 120 SOUTH LASALLE STREET, SUITE 1600, CHICAGO, IL, 60603-3406, US

(21) Appl. No.: 12/275,742

(22) Filed: Nov. 21, 2008

Related U.S. Application Data
(63) Continuation of application No. 10/872,826, filed on Jun. 21, 2004.
(60) Provisional application No. 60/479,778, filed on Jun. 19, 2003.

(51) Int. Cl. A61K 31/428; A61P 25/00

(52) U.S. Cl. 514/367

(57) ABSTRACT

Methods for inhibiting and/or reversing tau filament formation or fibrillization are provided. These methods can be used for treating certain neurological disorders in vivo by administering pharmaceutical compositions which inhibit and/or reverse tau filament formation or fibrillization. A preferred composition comprises 3-(2-hydroxyethyl )-2-[2-[[3-(2-hydroxyethyl )-5-methoxy-2-benzothiazolylidene]methyl]-1-butenyl]-5-methoxybenzothiazolium.

### FIELD OF THE INVENTION

The current invention relates to methods for inhibiting and/or reversing tau filament formation or fibrillization. This invention also relates to methods for treating certain neurological disorders in vivo by administering pharmaceutical compositions which inhibit and/or reverse tau filament formation or fibrillization.

### BACKGROUND

The microtubule-associated protein tau is a soluble cytosolic protein that is believed to contribute to the maintenance of the cytoskeleton (Johnson et al., Alzheimer's Disease Review 3: 125 (1998); Buee et al., Brain Research Reviews 33:95 (2000)). However, in many disease states, tau protein is induced by unknown cellular conditions to self-associate into filamentous structures (Spillantini et al., Trends Neurosci. 21: 428 (1998)). These filamentous forms of tau can be found in such varied neurodegenerative disorders such as Alzheimer's disease (AD) (Wood et al., Proc. Natl. Acad. Sci. USA 83: 4040 (1986); Kosik et al., Proc. Natl. Acad. Sci. U.S.A 83: 4044 (1986); Grundke-Iqbal et al., J. Biol. Chem. 261: 6084 (1986)), corticobasal degeneration (CBD) (Feany et al., Am. J. Pathol. 146: 1388 (1995)), progressive supranuclear palsy (PSP) (Tabaton et al., Ann. Neurol. 24: 407 (1988)), Pick's disease (PD) (Murayama et al., Ann. Neurol. 27: 394 (1990)), Down syndrome (Papasozomenos et al., Lab Invest. 60: 123 (1989)), and frontotemporal dementias and Parkinsonism linked to chromosome 17 (FTDP-17) (Spillantini et al., Proc. Natl. Acad. Sci. USA 94: 4113 (1997)). There remains a need for the identification of effective therapies for these neurodegenerative disorders.

Neuritic plaques, neurofibrillary tangles, and neuropil threads are hallmark lesions of Alzheimer's disease (AD) that contain filamentous intraneuronal inclusions of tau protein (Buee et al., Brain Res. Rev. 33: 95-130 (2000)). Because tau filaments form in brain regions associated with memory retention, and because their appearance correlates well with the degree of dementia, they have emerged as robust markers of disease progression (Braak et al., Acta. Neuropathol. (Berl) 87: 554-567 (1991); Braak et al., Acta Neuropathol. (Berl) 87: 554-567 (1994)). Tau filaments also appear in other neurodegenerative tauopathies, including Pick's disease and corticobasal degeneration, with the neuronal populations affected being disease dependent (Feany et al., Ann. Neurol. 87: 554-567 (1996)). Thus tau filament formation heralds the onset of cytoskeletal disorganization that is characteristic of degenerating neurons, and may represent a fundamental pathobiological response of neurons to various insults.

Genetic studies have extended these observations by establishing a direct link between certain neurodegenerative disorders and mutations in the tau gene (Spillantini et al., Neurogenetics 2: 193-205 (2000)). These autosomal-dominant dementias, such as FTDP-17, fall into several classes. One class consists of point mutations within the coding sequence of tau protein. A second class consists of intronic mutations that affect the distribution of alternatively spliced tau isoforms found in the insoluble tau deposits of these disorders. Each of the resultant “tauopathies” accumulates filamentous tau inclusions (Spillantini et al., Neurogenetics 2: 193-205 (2000)), as do transgenic mice harboring the FTDP-17 mutation P301L gene (Lewis et al., Nat. Genet. 25: 402-405 (2000); Gotz et al., J. Biol. Chem. 276: 529-534)). These findings emphasize the importance of tau protein in normal neuronal function and show that changes in tau structure can lead directly to filament formation and neurodegeneration.

In fact, merely overexpressing human tau in lamprey reticulospinal neurons is sufficient to drive filament accumulation and subsequent neuronal death (Hall et al., Am. J. Pathol 158: 235-246 (2001)). In the lamprey system, neurons continue to function until a critical mass of tau filaments is present. Overexpression of other polymerizing proteins, such as the neurofilament protomer NF180, also leads to filament formation but not neurodegeneration (Hall et al., Cell. Motil. Cytoskeleton 46: 166-182 (2000)). These data suggest that the assembly of tau protein into filamentous forms leads to a toxic gain of function for tau that exacerbates or potentially mediates degeneration in affected neurons.

Confirming these findings in vitro has been challenging because purified recombinant tau preparations do not polymerize spontaneously at physiological concentrations (low micromolar) and temperatures (King et al., Biochemistry 38: 14851-14859 (1999)). However, efficient formation of tau filaments with straight morphology from full-length tau protein can be induced in a matter of hours by the addition of fatty acids at 50 to 100 μM concentrations (King et al., Biochemistry 38: 14851-14859 (1999); Wilson et al., Am. J. Pathol 150: 2181-2195 (1997)). These agents act by forming micelles and presenting a negatively charged surface to tau protein. On the basis of seeding experiments, fatty acid-induced synthetic straight filaments are closely related to paired helical filaments (PHF) found in AD, and appear to correspond to a single hemifilament (King et al., Biochemistry 38: 14851-14859 (1999)). Using this paradigm, it has been shown that the rate and extent of tau fibrillization is influenced by C-terminal truncation and phosphorylation mimicry at residues S^{396/404}. It has also been shown that point mutations at known FTDP-17 sites, such as P301L, markedly promote tau filament formation (Gamblin et al., Biochemistry 39: 6136-6144 (2000); Abraha et al., J. Cell. Sci. 113: 3737-3745 (2000)). Thus, many of the tau modifications or mutations associated with filament formation and disease can be shown to accelerate tau fibrillization in vitro.

Using methods described in co-pending U.S. application Ser. No. 09/919,475, filed on Jul. 21, 2001, specific relatively low molecular weight ligands (generally less than about 400 daltons) have been identified which inhibit and/or reverse tau filament formation or fibrillization at substoichiometric concentrations relative to tau protomer. This co-pending application, which is owned by the same assignee of the present application, is hereby incorporated by reference in its entirety. These ligands or inhibitors can be used therapeutically to treat certain neurological disorders or disease states in vivo, including Alzheimer's disease, in which tau filaments are formed.

### SUMMARY OF THE INVENTION

In one embodiment, the present invention provides a method for regulating the assembly of the protein tau in the brain of a patient, comprising:

identifying a patient in need of a method for inhibiting tau fibrillization in the brain; and

administering to the patient a pharmacologically effective amount of an inhibitor of tau fibrillization, wherein the inhibitor is a compound of the general formula (Formula I)

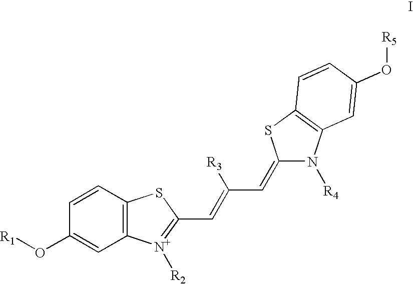

wherein R₁, R₃, and R₅ are independently an aliphatic radical having 1 to 6 carbon atoms and R₂ and R₄ are independently a second aliphatic radical having 1 to 6 carbon atoms or a hydroxyl-substituted aliphatic radical having one to six carbon atoms.

In one preferred embodiment, the inhibitor is 3-(2-hydroxyethyl)-2-[2-[[3-(2-hydroxyethyl)-5-methoxy-2-benzothiazolylidene]methyl]-1-butenyl]-5-methoxybenzothiazolium (N744), having the formula (Formula II)

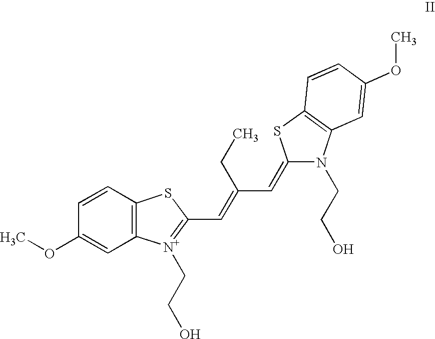

In one embodiment, the patient is a human. Generally the inhibitor is administered in an effective amount which can be determined using conventional techniques. Generally, the inhibitor is administered in an amount selected from about 10 mg per day to about 1000 mg per day. In one embodiment, the administering is performed repeatedly over a period of at least one week. In one embodiment, the administering is performed repeatedly over a period of at least one month. In one embodiment, the administering is performed repeatedly over a period of at least three months. In one embodiment, the administering is performed repeatedly over a period of at least one year. In another embodiment, the administering is performed at least once monthly. In another embodiment, the administering is performed at least once weekly. In another embodiment, the administering is performed at least once daily. In another embodiment, the administering is performed at least once weekly for at least one month. In another embodiment, the administering is performed at least once per day for at least one month.

### BRIEF DESCRIPTION OF THE DRAWINGS

FIG. 1. N744 inhibits tau fibrillization. Htau40 (4 μM) was incubated with arachidonic acid (75 μM) without agitation for 3.5 hours at 37° C. Aliquots were then stained with uranyl acetate and viewed in a transmission electron microscope as described in the Examples. FIG. 1A: In the presence DMSO (dimethylsulfoxide) vehicle control, htau40 formed abundant filaments with number average length of 111±6 (standard deviation) nm. FIG. 1B: In the presence of 4.1 μM N744, tau fibrillization was greatly inhibited.

FIG. 2. N744 inhibits tau fibrillization at substoichiometric concentrations. Htau40 (4 μM) was incubated (3.5 hours at 37° C.) with arachidonic acid (75 μM) in the presence of varying concentrations (0, 0.12, 0.41, 1.2, and 4.1 μM) of N744. Aliquots were then examined by transmission electron microscopy at 22,000-fold magnification. All filaments≧50 nm in length were measured from two negatives, summed, and plotted as total filament length (▪) and total filament number (□) versus N744 concentration in Hill plot format, where Y is the percent control filament length or filament number. Each line represents linear regression analysis of data points. Both total filament length and total filament number decreased in the presence of N744, with IC₅₀ values of 294±23 and 272±17 nM, respectively. Both Hill plots had a positive slope, with values of 1.84±0.14 and 1.61±0.10, respectively.

FIG. 3. N744 inhibits both tau filament nucleation and elongation. Htau40 (4 μM) was incubated (3 hours at 37° C.) in the presence of DMSO vehicle only (▪), or 0.12 (□), 0.41 (), 1.2 (◯), and 4.1 (▴) μM N744 and then examined by transmission electron microscopy at 22,000-fold magnification. Lengths and numbers of filaments≧50 nm in length were then measured from digitized images, summed, and plotted. Each data point represents the percentage of all filaments analyzed in 3 to 5 negatives (derived from 3722, 2972, 1248, 379, and 92 individually measured filaments, respectively) that segregated into consecutive length intervals (25 nm bins), whereas each line represents the best fit of the data points to an exponential distribution. At low concentrations of N744 (≦410 nM), length distributions did not differ significantly from DMSO vehicle control, suggesting that N744 did not modulate filament extension under these conditions. In contrast, further elevations of N744 concentrations (≧1.2 μM) led to significant shortening of length distributions, suggesting that filament extension was inhibited at these higher concentrations.

FIG. 4. Timecourse of N744-mediated disaggregation. Filaments prepared (3.5 hours at 37° C.) from htau40 (4 μM) and arachidonic acid (75 μM) were split into two equal pools and further incubated in the presence of DMSO vehicle alone (▪) or 4.7 μM N744 (□) for 19 hours. Aliquots of each reaction were stopped at 0, 1, 3, 5, 9, 12, and 19 hours by the addition of glutaraldehyde. Filaments≧50 nm in length were analyzed by the quantitative electron microscopy assay. Each data point represents total filament length per field±standard deviation (n=5 observations). In the presence of DMSO vehicle alone, total tau filament length decreases slowly over time with a first order rate of 0.022±0.005 h⁻¹. In the presence of N744, however, total filament length per field decreased with an initial first order rate of 0.12±0.01 h⁻¹ and a net rate of 0.10±0.02 h⁻¹ when corrected for DMSO vehicle alone. After 19 hours incubation in the presence of 4.7 μM N744, total filament length had decreased to 13±2% of that observed in the vehicle only control.

FIG. 5. Length distribution of filaments during N744-mediated disaggregation. The relative length distributions of htau40 filaments≧50 nm arising from the experiment shown in FIG. 3 were calculated and plotted. Each data point represents the percentage of all filaments analyzed in five fields that segregated into consecutive length intervals (50 nm bins), whereas each line represents the best fit of the data points to an exponential distribution. At time 0 h (FIG. 5A, top panel), length distributions for treatment with DMSO vehicle control alone (▪) and 4.7 μM N744 (□) were indistinguishable. Total filament length per field decreased over time, however, so that by 19 hours (FIG. 5B, bottom panel) there were significantly fewer filaments in every bin of the N744-treated aliquot (◯) relative to the DMSO only control (). The maintenance of an exponential distribution with continually decreasing filament numbers is consistent with end-wise disaggregation of tau filaments and inconsistent with random filament breakage.

FIG. 6. N744 is selective for tau fibrillization. In FIG. 6A, Aβ₁₋₄₀ (amyloid β peptide) (20 μM) was incubated in assembly buffer in the presence of DMSO vehicle alone () or 4.1 μM N744 (◯) and followed for 5 hours by absorbance at 400 nm. The resultant data was plotted using the first order kinetic model of Naiki and Gejyo (Methods Enzymol. 309: 305-318 (1999)), where Aₜ is the absorbance at time t, and A_{∞} is the maximal absorbance achieved at equilibrium (>5 hours). Each solid line represents linear regression analysis of the data points, whereas the dotted and dashed lines correspond to t_{1/2} in the presence and absence of N744, respectively. The close similarity in the two curves shows that N744 did not appreciably modulate the extent or half-life of Aβ₁₋₄₀ fibrillization under these conditions.

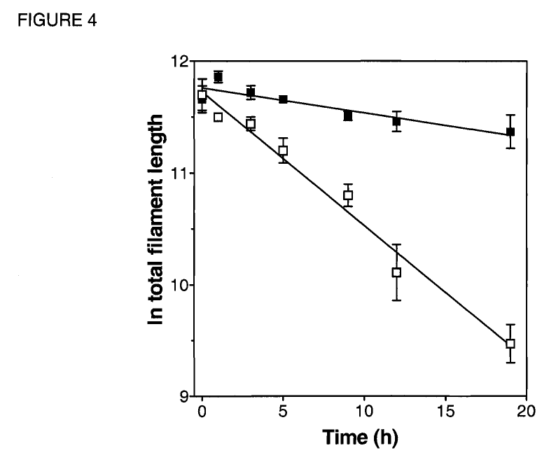

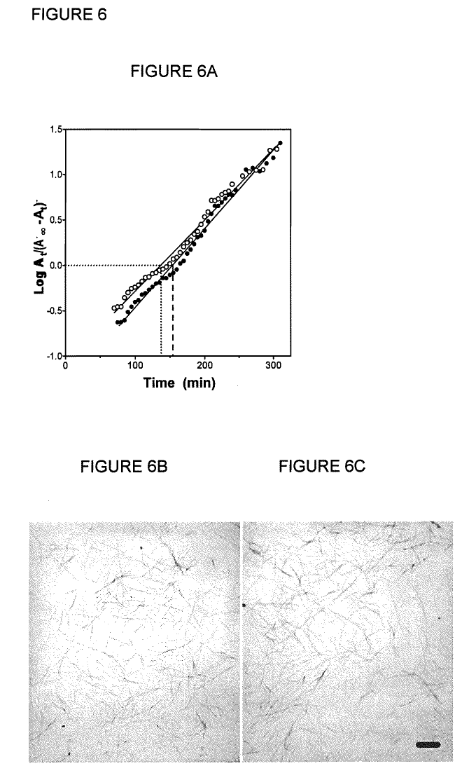

A second amyloid-forming protein, amylin, also was incubated in the presence of DMSO vehicle (FIG. 6B) or 4.1 μM N744 (FIG. 6C). Aliquots were removed over a 24 hour period and imaged by transmission electron microscopy. Images taken 3 hours after the initiation of the assembly process are shown. N744 did not interfere with amylin assembly under these conditions. Together these data suggest that N744 is selective for tau protein when assayed at substoichiometric concentrations. The bar in FIG. 6C indicates 500 nm.

### DESCRIPTION OF THE PREFERRED EMBODIMENTS

Alzheimer's disease is defined in part by the intraneuronal accumulation of filaments comprised of the microtubule associated protein tau. Because animal model studies suggest that a toxic gain of function accompanies tau fibrillization in neurons, selective pharmacological inhibitors of the process may slow neurodegeneration. The present invention provides small molecule inhibitors of tau fibrillization of Formula I

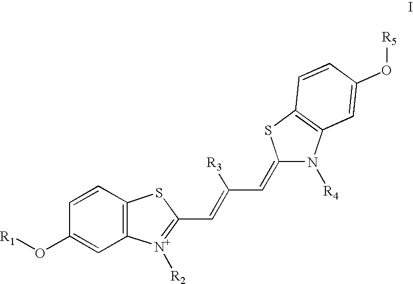

wherein R₁, R₃, and R₅ are independently an aliphatic radical having 1 to 6 carbon atoms and R₂ and R₄ are independently a second aliphatic radical having 1 to 6 carbon atoms or a hydroxyl-substituted aliphatic radical having one to six carbon atoms. In a preferred embodiment, the present invention also provides the inhibitor of tau fibrillization, 3-(2-hydroxyethyl)-2-[2-[[3-(2-hydroxyethyl)-5-methoxy-2-benzothiazolylidene]methyl]-1-butenyl]-5-methoxybenzothiazolium (referred to herein as N744), and shown in Formula II.

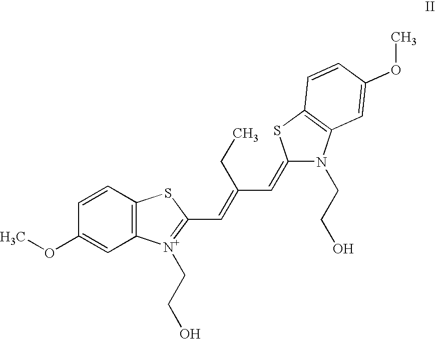

N744 is a benzenamine derivative broadly related to the Congo Red family of dyes in that it is planar and consists of two aromatic rings flanking a hydrocarbon linker. It is predicted to be positively charged at physiological pH.

N744 inhibits arachidonic acid induced fibrillization of full-length, four-repeat tau protein at substoichiometric concentrations relative to tau with an IC₅₀ below 300 nM. It also promotes tau disaggregation when added to mature synthetic filaments at concentrations stoichiometric with tau protomer. Disaggregation follows first order kinetics and is accompanied by a steady decrease in filament numbers, suggesting that N744 promotes endwise loss of tau molecules with limited filament breakage. Because of its activity in vitro, N744 may be useful for testing the tau hypothesis in cellular models of disease.

The data presented herein show that tau fibrillization can be inhibited by a compound of formula I, and specifically by N744, a small ligand (<400 Da) acting at substoichiometric concentrations relative to tau protomer and in the presence of >100-fold molar excess of fatty acid inducer. These data support the feasibility of antagonizing and even reversing tau filament formation in vivo.

The current invention includes a method for regulating the assembly of the protein tau in the brain of a mammal in need of such a regulation, wherein the method comprises administering to the mammal a pharmacologically effective amount of an inhibitor of tau fibrillization in a pharmaceutically-acceptable carrier. For purposes of this invention, the term “regulating the assembly of the protein tau” includes, but is not limited to, inhibiting and/or reversing tau filament formation or fibrillization and/or moderating the rate of tau filament formation or fibrillization.

Tau protein assembles into linear filaments capable of binding histochemical dyes such as Congo Red and thioflavin S, suggesting that tau protein polymerizes with the extended beta sheet conformation characteristic of “amyloid” deposits (Rochet et al., Curr. Opin. Struct. Biol. 10: 60-68 (2000); Serpell et al., J. Mol. Biol. 300: 1033-1039 (2000)). On the basis of ligand-mediated assembly reactions conducted in vitro with both fragmentary and full-length tau protein, fibrillization appears to be mediated by short hydrophobic sequences located in the microtubule repeat region (Abraha et al., J. Cell Sci. 113: 3737-3745 (2000); von Bergen et al., Proc. Nat'l. Acad. Sci. U.S.A. 97: 5129-5134 (2000)). However, sequences outside this region have a striking effect on both the kinetics of fibrillization and the organization of protomers within the filament (Abraha et al., J. Cell Sci. 113: 3737-3745 (2000); Giannetti et al., Protein Sci. 9: 2427-2435 (2000)). Thus, despite retaining general similarity with amyloid fibrils derived from other proteins, filaments of full-length tau protein offer potentially unique pharmacophores for binding polymerization inhibitors.

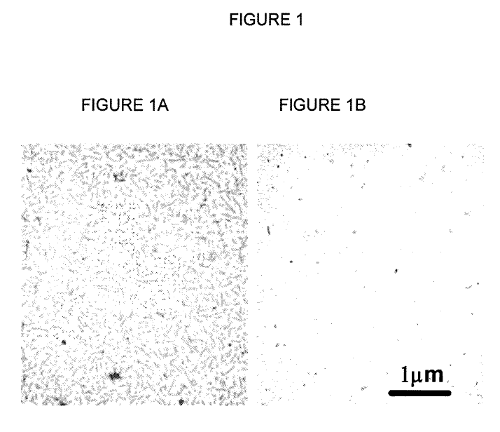

N744 appears to inhibit fatty-acid mediated formation of filaments from purified, recombinant htau40. The ability of small molecules to antagonize amyloid fibril formation has been reported previously (Lorenzo et al., Proc. Natl. Acad. Sci. U. S. A. 91: 12243-12247 (1994); Rudyk et al., J. Gen. Virol. 81: 1155-1164 (2000)). It has been postulated that these inhibitors act at different stages of assembly to either lower the effective monomer concentration, block growth at filament ends, or increase the rate of filament breakage (Masel et al., Biophys. Chem. 88: 47-59 (2000)). In the case of N744, its inclusion in tau assembly assays leads to a concentration dependent decrease in total tau filament mass (which is proportional to total length). The IC₅₀ for this effect was ˜300 nM. Assuming a mass per unit length value of 74.6 kDa/nm (King et al., J. Pathol. 158: 1481-1490 (2001)), ˜40% conversion of 4 μM tau to filamentous forms (Chirita et al., J. Biol. Chem. 278: in press (2003)), and a number average filament length of 111 nm in control reactions containing DMSO vehicle alone (FIG. 1A) yields ˜10 nM as an estimate of filament number concentration. Thus N744 inhibits tau fibrillization at concentrations substoichiometric with respect to tau protomer but well above the final concentration of filaments and therefore nuclei.

Inhibition of arachidonic acid-mediated nucleation appears to make a major contribution to N744 activity near the IC₅₀ because the IC₅₀ values for inhibition of total filament number and length were very similar. At concentrations approaching stoichiometry with total tau protomer, however, the effect of N744 on filament length distributions becomes apparent. Moreover, treatment of mature filaments with stoichiometric concentrations of N744 leads to filament diaggregation with first order kinetics, maintenance of a near exponential distribution of filament lengths, and to steadily decreasing numbers of filaments. These characteristics are consistent with progressive endwise disaggregation and inconsistent with catastrophic filament breakage along the filament length (Kristofferson et al., J. Biol. Chem. 255: 8567-8572 (1980)). Together with N744-mediated decreases in filament length distributions, these data suggest that stoichiometric concentrations of N744 affect the equilibrium between fibrillar and nonfibrillar tau so that dissociation of tau from filament ends predominates. As a result of the new equilibrium, fibrillization of 4 μM htau40 was no longer supported.

The pathway for tau fibrillization from recombinant monomer is not entirely clear but appears to parallel that of other amyloids by following the general scheme (Scheme I):

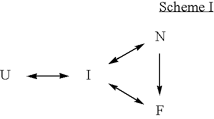

where U represents the unfolded state, I represents intermediate forms that may contain secondary or oligomeric structures such as dimers (Barghorn et al., Biochemistry 41: 14885-14896 (2002)), N represents the nucleus, the formation of which is rate limiting, and F represents filamentous forms, which may be multiple and include protofilaments (Uversky et al., J. Biol. Chem. 276: 10737-10744 (2001)). Mature filaments eventually reach equilibrium with nonfibrillar protein, presumably in its U and I forms, which is reflected in the critical concentration of assembly. Arachidonic acid accelerates this pathway by interacting with unfolded tau to form anionic micelles (Chirita et al., J. Biol. Chem. 278: in press (2003)). The resultant complexes nucleate very rapidly and produce large quantities of filaments at the low micromolar tau concentrations that normally yield few if any filaments in the absence of inducer (King et al., Biochemistry 38: 14851-14859 (1999); (Chirita et al., J. Biol. Chem. 278: in press (2003)). In experiments with α-synuclein, another amyloid forming protein (Spillantini et al., Proc. Natl. Acad. Sci. U S A 95: 6469-6473 (1998)), anionic micelles appear to induce fibrillization by shifting the equilibrium in favor of partially folded intermediate forms, resulting in shortened assembly lag times, increased apparent first order rates of assembly, and decreased critical concentrations at equilibrium relative to reactions conducted in the absence of micelles. Assuming that micelle-mediated fibrillization of tau retains these features, N744 appears to antagonize the action of arachidonic acid: it inhibits tau filament nucleation and appears to raise the critical concentration of assembly. This behavior probably does not derive from direct inhibition of arachidonic acid micellization, because although N744 is positively charged, and presumably able to interact with anionic micelles, its IC₅₀ for inhibition of tau fibrillization is <0.01% the molar concentration of arachidonic acid. In fact, diffuse cations such N744 typically depress the critical micelle concentration of anionic surfactants (Moroi et al., J. Colloid. Interface Sci. 198: 180-188 (1985)).

A more likely mechanism is suggested by the structural similarity between N744 and Congo Red. Like N744, Congo Red is a planar aromatic dye, and on the basis of its binding stoichiometry and optical properties (birefringence) is thought to bind all along the length of amyloid fibrils (Klunk et al., J. Histochem. Cytochem. 37: 1273-1281 (1989)). However, Congo Red also binds globular proteins and the secondary structure elements of partially folded intermediates (Khurana et al., J. Biol. Chem. 276: 22715-22721 (2001)). Compounds capable of binding globular monomers such as flufenamic acid acting on transthyretin, or colchicine acting on tubulin can lead to substoichiometric inhibition of aggregation similar to that described here for tau protein (Skoufias et al., Biochemistry 31: 738-746 (1992); Peterson et al., Proc. Natl. Acad. Sci. U.S.A. 95: 12956-12960 (1998)). In the latter example, substoichiometric inhibition of aggregation and promotion of disassembly depends upon filament polarity, where assembly and drug action occur primarily at one end while disassembly proceeds at the opposite end (Perez-Ramirez et al., Biochemistry 35: 3277-3285 (1996)). Because seeding experiments are consistent with tau filaments having growth polarity (King et al., Biochemistry 38: 14851-14859 (1999)), this mechanism cannot be ruled out at present. But because recombinant tau monomer is mostly random coil (Schweers et al., J. Biol. Chem. 269: 24290-24297 (1994)), it would appear unlikely that N744 interacts with tau in this way. Rather, N744 may bind an assembly competent intermediate to form an assembly incompetent aggregate as suggested for Congo Red (Khurana et al., J. Biol. Chem. 276: 22715-22721 (2001)). The resultant shift in equilibrium would be expected to lower the effective concentration of intermediate, resulting in slower filament nucleation. The relationship between filament nucleation rate and protein concentration has been proposed as:

dC/dt=kₙ(Pᵢ)ⁿ

where C is the number concentration of filaments, kₙ is the nucleation rate constant, Pᵢ is the concentration of assembly competent intermediate, and n is the number of molecules in the nucleus (Tobacman et al., J. Biol. Chem. 258: 3207-3214 (1983)). Thus small, N744-mediated changes in the concentration of an assembly competent intermediate are predicted to have large, non-linear effects on nucleation rate. The cooperative inhibition of tau filament nucleation with respect to N744 concentration (observed Hill coefficients between 1.6 and 1.8) may stem from this relationship. An inhibitor-mediated shift in equilibrium toward a fibrillization incompetent intermediate would also be expected to decrease the fibrillization rate and increase the amount of non-fibrillar protomer at equilibrium (Naiki et al., Biochemistry 36: 6243-6250 (1997)). The endwise disaggregation induced by N744 and the first order rate of approach to the new equilibrium are consistent with this model.

The close correlation between the spatial and temporal distributions of neurofibrillary lesions and the severity of neuronal cell loss and dementia suggests a central role for tau fibrillization in the development of AD (Braak and Braak, Acta. Neuropathol. (Berl) 82: 239-259 (1991); Gomez-Isla et al., J. Neurosci 16: 4491-4500 (1996); Ghoshal et al., Exp. Neurol. 177: 475-493 (2002)). This hypothesis has been greatly strengthened by the discovery of familial forms of neurofibrillary dementias that feature the development of neurofibrillary lesions in the absence of Aβ deposition and that are genetically linked to mutations in the tau gene (Hutton et al., Nature 393: 702-705 (1998); Spillantini et al., Proc Natl Acad Sci USA 95: 7737-7741 (1998)). Yet whether tau fibrillization represents a toxic gain of function (i.e., a metabolic disruption or toxicity caused by the filaments themselves) or loss of function (i.e., interference with normal tau functions via the sequestration of tau into filaments) has not been established. Studies on the functional characteristics of tau mutants associated with familial neurofibrillary dementias are equivocal; while some of these mutants exhibit decreased microtubule binding in cell culture (Hasegawa et al., FEBS Lett. 437: 207-210 (1998); Hong et al., Science 282: 1914-1917 (1998)), they also exhibit an increased tendency to form filaments in vitro (Goedert et al., FEBS Lett. 450: 306-311 (1999); Gamblin et al., Biochemistry 39: 6136-6144 (2000)). The autosomal dominant mode of inheritance of most familial neurofibrillary dementias (Reed et al., J Neuropathol Exp Neurol 57: 588-601 (1998)) suggests, but does not require, a “gain of function” mode of action, and it is possible that multiple tau-based mechanisms contribute to the neurodegeneration seen in the AD and the familial neurofibrillary dementias. A pharmacological approach to the problem using N744 may clarify the contribution of tau fibrillization to neurodegeneration. Its substoichiometric mode of action suggests that inhibition of tau fibrillization will be feasible even at the high tau concentrations found associated with neuritic lesions (Khatoon et al., J. Neurochem. 59: 750-753 (1992)).

On the basis of morphology and protomer stoichiometry, synthetic tau filaments induced by arachidonic acid treatment resemble straight filaments found early in disease (Perry et al., J. Neurosci. 11: 1748-1755 (1991)), and correspond to one hemifilament of authentic paired helical filaments (PHF) (King et al., Biochemistry 38: 14851-14859 (1999); King et al., Am. J. Pathol. 158: 1481-1490 (2001)). The apparent commonality in protomer organization among these morphologies suggests that N744 may be useful for modulating tau fibrillization in various cell and animal models of tauopathic neurofibrillary degeneration.

The dye 3-(2-hydroxyethyl)-2-[2-[[3-(2-hydroxyethyl)-5-methoxy-2-benzothiazolylidene]methyl]-1-butenyl]-5-methoxybenzothiazolium (N744) (Formula II), as well as similar compounds, has been found to inhibit fatty-acid mediated formation of straight filaments from purified, recombinant htau40.

The inhibitors suitable for use in the present invention are compounds of the general formula (Formula I)

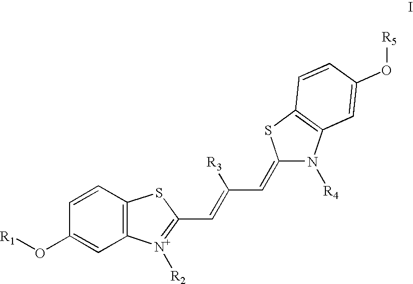

wherein R₁, R₃, and R₅ are independently an aliphatic radical having 1 to 6 carbon atoms and R₂ and R₄ are independently a second aliphatic radical having 1 to 6 carbon atoms or a hydroxyl-substituted aliphatic radical having one to six carbon atoms.

In one preferred embodiment, the inhibitor is 3-(2-hydroxyethyl)-2-[2-[[3-(2-hydroxyethyl)-5-methoxy-2-benzothiazolylidene]methyl]-1-butenyl]-5-methoxybenzothiazolium (N744), having the formula (Formula II)

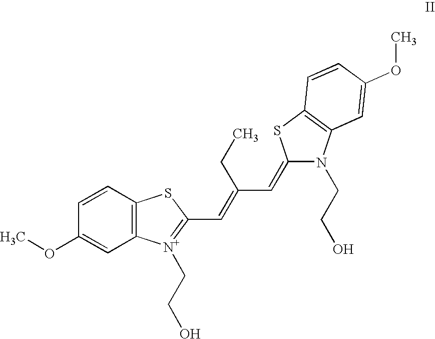

In Formula I above, R₁, R₃, R₅, are independently alkyl radicals having 1 to 6 carbon atoms. Examples of such alkyl or aliphatic radicals are methyl, ethyl, propyl, butyl, pentyl, and hexyl, including both straight and branched radicals. Preferably the alkyl radicals are methyl or ethyl and more preferably methyl.

In Formula I above, R₂ and R₄ are independently alkyl or aliphatic radicals having 1 to 6 carbon atoms or hydroxyl-substituted alkyl or aliphatic radicals having 1 to 6 carbon atoms. Examples of such alkyl or aliphatic radicals are methyl, ethyl, propyl, butyl, pentyl, and hexyl radicals, including both straight and branched radicals. Preferably the alkyl radicals are methyl or ethyl and more preferably methyl. Examples of such hydroxyl-substituted alkyl or aliphatic radicals are hydroxyl-substituted methyl, ethyl, propyl, butyl, pentyl, and hexyl, including both straight and branched radicals. Preferably the hydroxyl-substituted alkyl radicals are —(CH₂)ₙCH₂OH radicals where n is an integer 0 to 5; more preferably n is 1.

Inhibitors of Formula I were identified using essentially the same methods described in co-pending U.S. application Ser. No. 09/919,475, filed on Jul. 21, 2001. These inhibitors are specific relatively low molecular weight ligands which inhibit and/or reverse tau filament formation or fibrillization. This co-pending application, which is owned by the same assignee of the present application, is hereby incorporated by reference in its entirety. These ligands or inhibitors can be used therapeutically to treat certain neurological disorders or disease states, including Alzheimer's disease, in which tau filaments are formed.

In one especially preferred embodiment, the mammal is a human. Generally the inhibitor is administered in an effective amount which can be determined using conventional techniques. Generally, the inhibitor is administered in an amount selected from about 10 mg per day to about 1000 mg per day.

In one embodiment, the administering of the inhibitors of this invention is performed repeatedly over a period of at least one week. In one embodiment, the administering is performed repeatedly over a period of at least one month. In one embodiment, the administering is performed repeatedly over a period of at least three months. In one embodiment, the administering is performed repeatedly over a period of at least one year. In another embodiment, the administering is performed at least once monthly. In another embodiment, the administering is performed at least once weekly. In another embodiment, the administering is performed at least once daily. In another embodiment, the administering is performed at least once weekly for at least one month. In another embodiment, the administering is performed at least once per day for at least one month.

This aspect of the invention provides for treatment and/or prevention of various diseases and disorders associated with tau fibrillization. The invention provides methods of treatment (and prophylaxis) by administration to a subject of an effective amount of a therapeutic of the invention. In a preferred aspect, the therapeutic is substantially purified. The patient or subject is preferably an animal, including, but not limited to, cows, pigs, horses, chickens, cats, dogs, and the like, and more preferably is a mammal, and most preferably is a human.

Various delivery systems are known and can be used to administer a therapeutic of the invention. Such systems include, for example, encapsulation in liposomes, microparticles, microcapsules, recombinant cells capable of expressing the therapeutic (see, e.g., Wu and Wu, “Receptor-mediated in vitro gene transformation by a soluble DNA carrier system,” J. Biol. Chem. 262:4429 (1987)), construction of a therapeutic nucleic acid as part of a retroviral or other vector, and the like. Methods of introduction include, but are not limited to, intradermal, intramuscular, intraperitoneal, intravenous, subcutaneous, intranasal, epidural, and oral routes. The therapeutics may be administered by any convenient route, including, for example, infusion or bolus injection, absorption through epithelial or mucocutaneous linings (e.g., oral mucosa, rectal and intestinal mucosa, and the like) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed (e.g., by an inhaler or nebulizer) using a formulation containing an aerosolizing agent.

In a specific embodiment, it may be desirable to administer the pharmaceutical compositions of the invention locally to the area in need of treatment, such as the brain. This may be achieved by, for example, and not by way of limitation, local infusion during surgery, topical application (e.g., wound dressing), injection, catheter, suppository, or implant (e.g., implants formed from porous, non-porous, or gelatinous materials, including membranes, such as sialastic membranes or fibers), and the like. In one embodiment, administration can be by direct injection at the site (or former site) of a tissue that is subject to damage by oxidation, such as the brain. In another embodiment, the therapeutic can be delivered in a vesicle, in particular a liposome (see, e.g., Langer, “New methods of drug delivery,” Science 249:1527 (1990); Treat et al., in Liposomes in the Therapy of Infectious Disease and Cancer, Lopez-Berestein and Fidler (eds.), Liss, N.Y., pp. 353-365 (1989)).

In yet another embodiment, the therapeutic can be delivered in a controlled release system. In one embodiment, a pump may be used (see, e.g., Langer, (1990); Sefton, “Implantable pumps,” Crit. Rev. Biomed. Eng. 14: 201 (1987); Buchwald et al., “Long-term, continuous intravenous heparin administration by an implantable infusion pump in ambulatory patients with recurrent venous thrombosis,” Surgery 88: 507 (1980); and Saudek et al., “A preliminary trial of the programmable implantable medication system for insulin delivery,” N. Engl. J. Med. 321: 574 (1989)). In another embodiment, polymeric materials can be used (see, e.g., Ranger et al., Macromol. Sci. Rev. Macromol. Chem. 23: 61 (1983); Levy et al., “Inhibition of calcification of bioprosthetic heart valves by local controlled-release diphosphonate,” Science 228:190 (1985); During et al., “Controlled release of dopamine from a polymeric brain implant: in vivo characterization,” Ann. Neurol. 25: 351 (1989); and Howard et al., “Intracerebral drug delivery in rats with lesion-induced memory deficits,” J. Neurosurg. 71: 105 (1989)). Other controlled release systems discussed in the review by Langer et al. (1990) can also be used.

Generally the inhibitors of this invention typically are administered using a pharmaceutically acceptable carrier. The term “pharmaceutically acceptable” means approved by a regulatory agency of the federal or a state government or listed in the U.S. Pharmacopeia or other generally recognized pharmacopeia for use in animals, and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic, if desired, can also contain minor amounts of wetting or emulsifying agents, or pH buffering agents. These therapeutics can take the form of solutions, suspensions, emulsion, tablets, pills, capsules, powders, sustained-release formulations, and the like. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Examples of suitable pharmaceutical carriers are described in “Remington's Pharmaceutical Sciences” by E. W. Martin. Such therapeutics will contain a therapeutically effective amount of the active ingredient, preferably in purified form, together with a suitable amount of carrier so as to provide proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampule of sterile water or saline can be provided so that the ingredients may be mixed prior to administration.

The amount of the therapeutic of the invention which will be effective depends on the nature of the tau-related disorder or condition, as well as the stage of the disorder or condition. Effective amounts can be determined by standard clinical techniques. In addition, in vitro assays, such as those described below, may optionally be employed to help identify optimal dosage ranges. The precise dose to be employed in the formulation will also depend on the route of administration, and should be decided according to the judgment of the health care practitioner and each patient's circumstances. However, suitable dosage ranges are about 10 mg/day to about 1000 mg/day. Effective doses may be extrapolated from dose-response curves derived from in vitro or animal model test systems. The invention also provides a pharmaceutical pack or kit comprising one or more containers filled with one or more of the ingredients of the therapeutics of the invention.

In one embodiment, the method for regulating the assembly of the protein tau in the brain of a patient comprises: identifying a patient in need of a method for inhibiting tau fibrillization in the brain; and administering to the patient a pharmacologically effective amount of an inhibitor of tau fibrillization of formula I or II as defined herein.

In one embodiment, the identifying being based on identifying mutant genomic subtypes of tau in the patient. Typically, these mutant subtypes are involved with increased Tau protein fibrillization. See review by Spillantini et al., Trends in Neurosciences, 21: 428 (1998). In another embodiment, the identifying is other than a diagnosis of Alzheimer's disease. For this embodiment, the identifying may be, but is not limited to, the diagnosis of another disorder involving tau fibrillization, such as Pick's disease, progressive supranuclear palsy, corticobasal degeneration and familial frontotemporal dementia, and parkinsonism linked to chromosome 17 (FTDP-17).

The following examples describe and illustrate the methods and compositions of the invention. These examples are intended to be merely illustrative of the present invention, and not limiting thereof in either scope or spirit. Unless indicated otherwise, all percentages are by weight. Those skilled in the art will readily understand that variations of the materials, conditions, and processes described in the example can be used.

### EXAMPLES

General Experimental Procedures

Materials. Recombinant polyhistidine-tagged htau40 was expressed and purified as described previously (Gamblin et al., Biochemistry 39: 14203-14210 (2000); Carmel et al., Biol. Chem. 271: 32789-32795)). Human Aβ₁₋₄₀ (Bachem; Philadelphia, Pa.) was dissolved in DMSO (500 μM), sonicated (30 minutes at room temperature) and filtered (0.2 μM cutoff) before use. Stock solutions of human amylin (Bachem; Philadelphia, Pa.) were prepared in water (250 μM). Arachidonic acid (Fluka; Milwaukee, Wis.) was dissolved in 100% ethanol and stored under argon gas at −80° C. until used. Tau fibrillization inhibitor 3-(2-hydroxyethyl )-2-[2-[[3-(2-hydroxyethyl )-5-methoxy-2-benzothiazolylidene]methyl]-1-butenyl]-5-methoxybenzothiazolium (Neuronautics, Inc.; Evanston, Ill.) was dissolved in DMSO (10 mM stock) and stored at −20° C.

Tau Aggregation. Purified recombinant htau40 was polymerized as described previously (King et al., Biochemistry 38: 14851-14859 (1999); Wilson et al., Am. J. Pathol 150: 2181-2195 (1997); King et al., J. Neurochem 74: 1749-1757 (2000)). Under standard conditions, 4 μM (final concentration) htau40 was incubated with arachidonic acid in Assembly Buffer (10 mM 4-[2-hydroxyethyl]-1-piperazineethanesulfonic acid, 100 mM NaCl, and 5 mM DTT) at either room temperature or at 37° C. Fibrillization was induced by the addition of arachidonic acid (75-100 μM) and continued for 3 to 6 hours until analyzed by electron microscopy as described below. When present, N744 final concentration varied between 0.12 and 4.1 μM in aggregation assays. (N744 final concentration of up to 4.7 μM was used in disaggregation assays (see below)). Control reactions were normalized for DMSO vehicle, which was limited to no more than 5% (v/v) in all reactions.

Tau Disaggregation. Solutions of purified htau40 (4 μM) were polymerized under standard conditions as described above for 3.5 hours, then divided into two separate tubes. One tube received N744 at a final concentration of 4.7 μM, whereas the second tube received DMSO vehicle alone. Aliquots were removed from each sample after 0, 1, 3, 5, 9, 12, and 19 hours incubation and subjected to the electron microscopy assay described below. Control (no N744) reactions were normalized for DMSO vehicle, which was kept below 5.7% (v/v) in all reactions.

Transmission Electron Microscopy. Aliquots (50 μl) of aggregation and disaggregation reactions were removed, fixed with glutaraldehyde (2%), and adsorbed (1 minute) onto 300 mesh formvar/carbon-coated copper grids (Electron Microscopy Sciences; Ft. Washington, Pa.). The resultant grids were washed with water, stained (1 minute) with 2% uranyl acetate (Electron Microscopy Sciences), washed again with water, blotted dry, and viewed in a Phillips CM 12 microscope operated at 65 kV. Three to five random images from each experimental condition were captured on film at 22,000× magnification, digitized, calibrated, and imported into Optimas 6.5.1 for quantitation of filament length and number as described previously (King et al., J. Neurochem 74: 1749-1757 (2000). An individual filament is defined as any object greater than 50 nm in its long axis. Filaments were counted manually. Filament counts are reported as an average±standard deviation for both total filament length and total filament number. Length distributions were quantified in 25 nm (assembly) or 50 nm (disassembly) wide bins.

Aβ₁₋₄₀ Aggregation. Aggregation was initiated by diluting the Aβ stock solution to 20 μM final concentration in aggregation buffer (150 mM NaCl, 10 mM 2-[N-morpholino]ethanesulfonic acid, pH 6.2; final volume 300 μl). Turbidity resulting from Aβ aggregation in the presence (4.1 μM final concentration) and absence of N744 was monitored as a function of time in a Beckman DU640B spectrophotometer at 400 nm versus a DMSO vehicle blank (Snyder et al., Biophys 67: 1216-1228 (1994); Evans et al., Proc. Natl. Acad. Sci U.S.A. 92: 763-767 (1995)). Cuvettes were vortexed before each reading. Total DMSO vehicle concentration was controlled among samples and did not exceed 6% (v/v).

Amylin Aggregation. Aggregation was initiated by diluting the peptide in 10 mM Tris-HCl, pH 7.3 to a final concentration of 50 μM (Goldsbury et al., J. Struct. Biol. 130: 352-362 (2000)) in the presence or absence of 4.1 μM N744. Aliquots were removed after 0, 1, 3, 5, 7, and 24 hours and prepared for EM as described above. Total DMSO vehicle concentration did not exceed 5% (v/v).

Analytical methods. Tau protein concentrations were determined by absorbance at 280 nm (Carmel et al., Biol. Chem. 271: 32789-32795 (1996)). All errors derived from linear regression analysis are 95% confidence limits unless otherwise noted.

### Example 1

Inhibition of tau Fibrillization. To identify chemical antagonists of tau fibrillization, a library of small molecules was screened for inhibitory activity against htau40 (2 μM) assembly induced by arachidonic acid (50 μM) under near-physiological conditions using a fluorescence-based assay (Wilson et al., Am. J. Pathol 150: 2181-2195 (1997)). The structure of N744, N744 is one inhibitor identified by the screen; its structure is shown in Formula I. It is a charged molecule (at physiological pH) and is broadly related to the Congo Red family of compounds in being a planar aromatic dye.

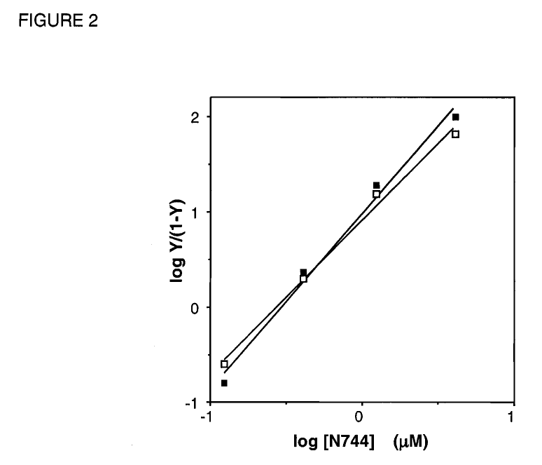

The ability of N744 to antagonize the fibrillization of htau40 (4 μM) induced by arachidonic acid (75 μM) under standard conditions was examined by transmission electron microscopy (King et al., Biochemistry 38: 14851-14859 (1999). Typically, approximately 50% of htau40 protomer is incorporated into filaments under these conditions. In the presence of DMSO vehicle alone, htau40 polymerized to form large numbers of filaments with straight morphology (FIG. 1A). In the presence of N744 (4.1 μM; approximately 1:1 molar stoichiometry with respect to tau protomer), however, fibrillization as reflected in either the total number or total length of all filaments was greatly inhibited (FIG. 1B). Varying N744 concentration between 0.124 and 4.1 μM revealed that inhibitory activity was graded, with filament formation as measured by total filament length inhibited with an IC₅₀ of 294±23 nM and a Hill slope of 1.84±0.14 (FIG. 2). These data confirmed that N744 was a potent inhibitor of tau fibrillization, being active at substoichiometric concentrations relative to tau protomer and arachidonic acid inducer.

### Example 2

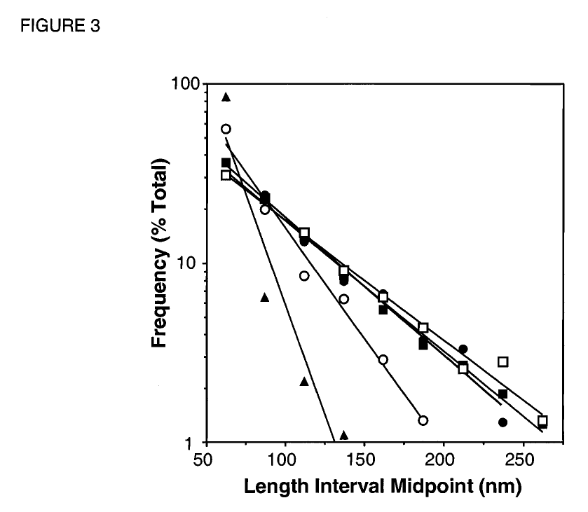

Inhibitory Mechanism. Tau fibrillization is characterized by nucleation and extension phases. To distinguish the effect of N744 on these two phases, filament length distributions were measured as a function of inhibitor concentration and compared to control reactions containing DMSO vehicle alone. The large number of filaments formed in the control reaction adopted an exponential length distribution (FIG. 3). This distribution was maintained at low N744 concentrations (i.e., near the IC₅₀; FIG. 3), but the number of filaments formed decreased relative to control reactions (FIG. 2). As N744 concentrations were increased to approach molar stoichiometry with htau40 protomer, still further decreases in filament numbers were observed (FIG. 2). These data suggest that a principal action of N744 is to inhibit tau filament nucleation. Indeed, the dose response curve for inhibition of tau filament number is nearly identical to the dose response curve for inhibition of total filament length (FIG. 2). Nonetheless, N744 at stoichiometric concentrations also shifted the filament length distribution toward shorter lengths relative to DMSO vehicle controls (FIG. 3). Thus N744 appears capable of inhibiting tau filament nucleation at substoichiometric concentrations but can inhibit both nucleation and extension as its concentration approaches molar stoichiometry with tau protomer.

### Example 3

N744 Promotes tau Filament Disaggregation. The ability of N744 to inhibit tau filament extension at near stoichiometric concentrations suggests that it may be capable of destabilizing mature filaments as well. To test this hypothesis, htau40 (4 μM) was polymerized with arachidonic acid (75 μM) over a 3.5 hour period after which time equal aliquots were treated with N744 (4.7 μM) or DMSO vehicle alone and filament numbers and lengths were measured over a 19 hour “chase” by electron microscopy. In the absence of N744, total htau40 filament length decreased 23±4% over this time period (FIG. 4). Because sample dilution was only 6% in the experimental paradigm, it appeared that DMSO alone destabilized tau filaments at these concentrations. In contrast, addition of N744 (4.7 μM) led to a more rapid decrease in total filament length so that 87±13% of total filament length was lost over the 19 hour time course. The initial rate of filament loss was well modeled as a first order decay (r²=0.981; k=0.12±0.01 h⁻¹) under these conditions (FIG. 4). These data suggest that N744 could destabilize mature filaments and decrease total filament length with first order kinetics at a net rate (i.e, the rate corrected for dilution and DMSO effect) of 0.10±0.02 h⁻¹.

### Example 4

Mechanism of Disaggregation. Filament dissaggregation may result from N744 promoting random filament breakage or by promoting endwise depolymerization (Masel et al., Biophys. Chem. 88: 47-59 (2000)). The kinetic characteristics of endwise depolymerization of linear protein assemblies at equilibrium depends on the length distribution of polymers (Kristofferson et al., J. Biol. Chem 255: 8567-8572 (1980)). For tau filaments, which adopt an exponential distribution of lengths (Gamblin et al., Biochemistry 39: 14203-14210 (2000); Wilson et al., J. Biol. Chem. 270: 24306-24314 (1995)) dissociation rates are predicted to be first order (Kristofferson et al., J. Biol. Chem 255: 8567-8572 (1980). Moreover, filament disassembly is predicted to proceed while maintaining an exponential distribution of gradually shortening filaments lengths (Kristofferson et al., J. Biol. Chem 255: 8567-8572 (1980)).

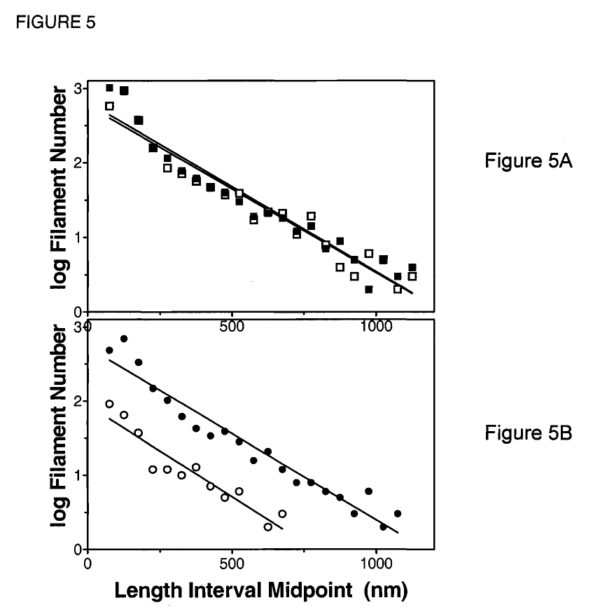

The observation of first order ligand-induced filament depolymerization suggested that N744 promoted sequential release of tau protomers from filament ends rather than by promoting random filament breakage. To confirm this hypothesis, the length distribution of tau polymers was examined as a function of time (19 hours) after treatment of preassembled tau filaments with N744 (4.7 μM) or DMSO vehicle alone. At time 0, both N744-treated and control reactions showed identical exponential distributions of tau filament lengths. Consistent with the endwise depolymerization model, exponential filament length distributions were maintained throughout the N744-mediated depolymerization reaction as the filaments shifted to shorter lengths relative to the control reaction (shown for time 0 and 19 hours only; FIG. 5). Moreover, N744-mediated depolymerization was accompanied by a slow decrease in the number of filaments greater than 50 nm in length (shown for time 0 and 19 hours only; FIG. 5), which was inconsistent with the random breakage-mediated depolymerization model. Together these data suggest that treatment of mature synthetic tau filaments with stoichiometric concentrations of N744 promotes endwise filament deaggregation.

### Example 5

Selectivity of tau Fibrillization Antagonism. Other dyes have been shown to bind a variety of amyloid aggregates at low micromolar concentrations, including those formed from Aβ and insulin (Caprathe et al., U.S. Pat. No. 6,001,331 (1999)). To determine whether amyloid binding was accompanied by fibrillization inhibitory activity, the ability of N744 to inhibit Aβ₁₋₄₀ assembly was examined. In the absence of ligand, Aβ₁₋₄₀ (20 μM) polymerized spontaneously after a lag of about 80 minutes. Plotting the reaction data using a first-order kinetic model described previously (Naiki et al., Methods Enzymol 309: 305-318 (1999)) yielded linear semilogarithmic plots of optical density vs. time and revealed a half-life of assembly of 136±3 minutes (FIG. 6). The presence of N744 at concentrations that were substoichiometric with respect to Aβ₁₋₄₀ protomer (4.1 μM) altered Aβ₁₋₄₀ assembly kinetics only modestly (t_{1/2}=153±3 minutes; FIG. 6), suggesting that N744 had little effect on Aβ₁₋₄₀ assembly under these conditions.

The activity of N744 on another amyloid-forming protein, amylin (Goldsbury et al., J. Struct. Biol. 130: 352-362 (2000)), was also examined. On the basis of qualitative electron microscopy analysis, the presence of 4.1 μM N744 did not modulate the fibrillization of 50 μM amylin over a 24 hour period (FIG. 6). These data confirm that, despite similarities in polymer structures (i.e., extended β-sheet) formed from different protein protomers, it is possible to select small ligands such as N744 with target-selective inhibitory activity at substoichiometric concentrations.

Throughout this application, various patents, publications, books, and nucleic acid and amino acid sequences have been cited. The entireties of each of these patents, publications, books, and sequences are hereby incorporated by reference into this application.

1-8. (canceled)

9. A method for reducing the assembly of the protein tau in the brain of a patient, said method comprising:
identifying a patient that is experiencing tau fibrillization in its brain; and
administering to the patient a pharmacologically effective amount of an inhibitor of tau fibrillization, wherein the inhibitor is a compound of the general formula I 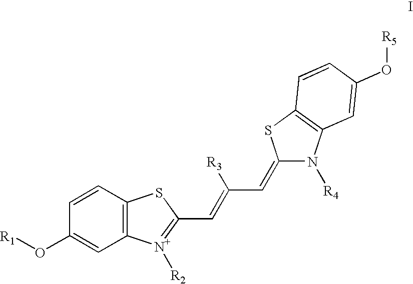
or a pharmaceutically acceptable salt thereof, wherein R₁, R₃, and R₅ are independently an aliphatic radical having 1 to 6 carbon atoms and R₂ and R₄ are independently a second aliphatic radical having 1 to 6 carbon atoms or a hydroxyl-substituted aliphatic radical having one to six carbon atoms.

10. The method of claim 1, wherein R₁, R₃, and R₅ are methyl radicals, and R₂ and R₄ are 2-hydroxyethyl radicals.

11. The method of claim 1, wherein the patient is a human and the pharmacologically effective amount is about 10 to about 1000 mg per day.

12. The method of claim 2, wherein the patient is a human and the pharmacologically effective amount is about 10 to about 1000 mg per day.

13. A method for inhibiting or reversing tau filament formation in the brain of a mammal, said method comprising:
identifying a mammal that is exhibiting tau fibrillization in its brain; and
administering to the mammal a pharmacologically effective amount of an inhibitor of tau fibrillization of formula II 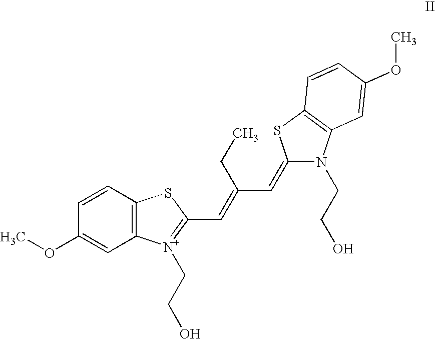
or a pharmaceutically acceptable salt thereof.

14. The method of claim 5, wherein the mammal is a human.

15. The method of claim 5, wherein the pharmacologically effective amount is about 10 to about 1000 mg per day.

16. The method of claim 6, wherein the pharmacologically effective amount is about 10 to about 1000 mg per day.

17. The method of claim 1 wherein the assembly of the protein tau in the patient's brain is linked to Alzheimer's disease.

18. The method of claim 5 wherein the tau fibrillization in the mammal's brain is linked to Alzheimer's disease.
